(19) United States
(12) Patent Application Publication
(10) Pub. No.: US 2008/0033278 A1
(43) Pub. Date: Feb. 7, 2008

(54) SYSTEM AND METHOD FOR TRACKING MEDICAL DEVICE USING MAGNETIC RESONANCE DETECTION

(75) Inventor: Benny Assif, Ramat Hasharon (IL)

Correspondence Address: VISTA IP LAW GROUP LLP, 12930 Saratoga Avenue, Suite D-2, Saratoga, CA, 95070, US

(73) Assignee: INSIGHTEC LTD., Tirat Carmel (IL)

(21) Appl. No.: 11/675,446

(22) Filed: Feb. 15, 2007

Related U.S. Application Data
(60) Provisional application No. 60/821,121, filed on Aug. 1, 2006.

(51) Int. Cl. A61B 5/055

(52) U.S. Cl. 600/410

(57) ABSTRACT

A system and method for using magnetic resonance to track a medical device which attenuates interfering MR signals. The system comprises an MR tracking device comprising one or more radiofrequency (RF) tracking coils adapted to receive a magnetic resonance MR response signal from nuclei excited by an RF pulse. Ideally, the RF tracking coil only receives an MR response signal from nuclei in matter in the close vicinity of the tracking device. The tracking device is attached to medical device. The interfering signal tends to emanate from large objects or large volumes that are outside the close vicinity of the tracking device but which are still weakly coupled to the tracking device. A dephasing gradient is applied perpendicular to the readout gradient before the RF response signal is received thereby dephasing the interfering signal emanating from remote nuclei, which strongly attenuates the interfering signal while the MR response signal is substantially unaffected. The system can also detect errors in tracking location by checking the amplitude of the signal from each coil and the detected distance between each coil, and correcting for such errors by ignoring data from coils having low amplitude or location deviating from known location relative to the other coils.

### RELATED APPLICATION DATA

This application claims the benefit under 35 U.S.C. 119 to U.S. Provisional Application No. 60/821,121, filed on Aug. 1, 2006, the contents of which are incorporated herein by reference.

### FIELD OF THE INVENTION

The field of the invention generally relates to systems and methods for monitoring the position of medical devices attached or inserted into a body during a medical procedure, and more particularly to tracking such medical devices using magnetic resonance imaging.

### BACKGROUND OF THE INVENTION

Magnetic resonance (MR) imaging of internal human tissue of the body and also MR tracking of devices inserted into the body is known by those of skill in the art, and therefore, need not be described in detail herein. Such MR imaging may be used for numerous medical procedures, including diagnosis, surgery, non-invasive surgery, minimally invasive surgery, and the like. In general terms, the method of imaging using MR consists of subjecting the subject matter to be imaged to a uniform magnetic field. The magnetic field causes the hydrogen nuclei spins to align and precess about the axial field of the magnetic field. The subject matter is then subjected to radiofrequency (RF) magnetic field pulses in a plane perpendicular to the static magnetic field. The RF pulse causes some of the aligned spins to alternate between a temporary non-aligned high-energy state and the aligned state, thereby emitting an RF response signal, called MR echo. A receiver detects the duration, strength, and source location of the MR echo signal and such data is then stored for processing. It is known that different tissues produce different RF response signals and this property is used to create contrast in an image.

In order to selectively image an entire area of interest, data is acquired utilizing magnetic field gradients (Gₓ, G_{y}, G_{z}). The data is then processed using known computational methods, such as Fourier transformations, to calculate the spatial location of the various tissue types and create an image of the subject matter of interest.

MR imaging is also used effectively to image during a medical procedure to assist in locating and guiding medical instruments used during the procedure. For example, a patient can be placed in an MR imaging (MRI) machine and then a medical procedure is performed using a medical instrument, such as a biopsy needle, catheter, ultrasonic ablating device or other instrument. The medical instruments may be for insertion into a patient or they may be devices that are used external to the patient which have a therapeutic effect on the patient or assist in diagnosis. For instance, the medical instrument can be an ultrasonic device, which is disposed external of the patient's body which focuses ultrasonic energy used to ablate tissue or other material on or within a patient's body. The MRI machine preferably produces images at a high rate so that the location of the instrument relative to the position of the patient and/or the patient's internal tissues may be monitored in real-time or substantially real-time. The MRI machine is used both, to image the surrounding body tissue as well as for locating the instrument such that the image and the overlaid instrument on the image may be used to track the absolute location of the instrument, as well as the location relative to the patient's body tissue.

Because medical instruments may not be directly detected by an MRI system precisely, or with high resolution, due to the shape, material, or other properties of the instrument, MR tracking techniques and devices have been developed. Several tracking systems have been described in U.S. Pat. Nos. 5,622,170; 5,617,857; 5,271,400; 5,318,025; and 6,289,233. The disclosures of each of the aforementioned U.S. patents are hereby incorporated by reference herein in their entireties. The tracking systems described in these U.S. patents comprise a tracking device which is attached to the instrument to be tracked, such as a catheter, needle or other device. The tracking device typically consists of small coil of wire (RF coil) which can receive MR response signals generated by subject matter (such as tissue) in response to the static magnetic field, gradient field and RF pulse of the MRI system. The RF coil is small such that the coil sensitivity drops fast away from the coil or in other words, it only detects the MR response signal from excited nuclei very close to the coil.

In use, a patient and a medical device with a tracking device are placed in the bore of an MRI system. A non-selective (meaning non-specific as to location within the bore) RF excitation pulse is then applied. The RF pulses causes an MR response signal, as described above, from the subject matter, including subject matter in the vicinity of the tracking coil. The tracking coil receives the MR response signal from the subject matter and this received signal is amplified, processed and digitally sampled before being transferred to the tracking processing system for analysis of the sampled data. Utilizing the known magnetic gradients of the MRI system, and the Larmor frequency properties of atomic nuclei spins, this data can be processed using computational methods known in the art to calculate the location of the tracking device, and therefore the location of the medical device to which the tracking device is attached. At the same time, the MRI system is used to spatially locate and image a location of interest in the patient. Then, the positional information (including an image, representation or illustration of the medical device) can be superimposed on the image of the location of interest (or any other image of interest created by the MRI system). The images can be displayed on any suitable display such as a computer monitor, a printout or other display format.

Ideally, the region of special sensitivity of the RF coil of the tracking device is so small that it only receives the MR response signal from the exact location of the coil. In this situation, the RF tracking coil does not receive any interfering signals from surrounding material or from other devices and equipment present in the MR bore. However, in practice, there may be significant interference received by the RF coil. For example, MR sensitive material which is located relatively remote from the RF coil emits an MR response signal that is at least weakly coupled to the RF coil. This signal represents interference which makes the tracking device less accurate because it is emitted by material that is not in close vicinity to the RF tracking coil. Other coils present in the MR bore are also receiving signals from the MR sensitive material that is located relatively far away from the RF tracking coil. These coils are magnetically coupled to the RF tracking coil, inducing interfering signals from remote material into the RF tracking coil. Moreover, although the coupling may be relatively weak, the signal can cause significant interference with the main signal because the interfering signal emanates from a volume of material that is many orders of magnitude larger than the primary signal emitted by the sensitive volume in close vicinity to the RF tracking coil.

Therefore, there is need for a system and method for locating and tracking a medical device using MR systems which overcomes the problems associated with prior systems.

### SUMMARY OF THE INVENTION

In accordance with the present invention, a system and method is described for using MR to track a medical device which reduces the interference signal suffered by prior systems. The system comprises an MR tracking device(s) which may comprise RF tracking coil or other suitable receiving component which can receive an MR response signal emitted by material excited by an MR machine. The RF tracking coil may comprise a conductive wire formed into a helical coil. A small tube filled with MR sensitive material like water or oil may be inserted into the winding of the RF tracking coil. The MR tracking device(s) may be attached to any medical device such as an ultrasonic or radiation ablation device, a catheter or other instrument.

In one innovative aspect of the present invention, an improved pulse sequence for the magnetic field gradient is utilized to attenuate the interfering signal suffered by prior systems. The interfering signal is primarily produced by a wide spread, large volume of MR sensitive material outside the close vicinity of the RF tracking coil. Therefore, the interfering signal, has a very wide bandwidth, and short echo duration. In comparison, the MR response signal emanates from the small volume in the vicinity of the RF tracking coil has a very low bandwidth and long echo duration.

By applying a dephasing gradient perpendicular to the readout gradient, preferably along the longest axis of the material causing the interfering signal, before applying the readout gradient, the interfering signal is dephased and as a result, strongly attenuated. At the same time, the MR response signal from the vicinity of the tracking RF coils is almost unaffected by the dephasing gradient because of its small physical size.

It thus is an object of the invention to provide a system and method for an MR tracking device which has reduces interference in the response signal.

### BRIEF DESCRIPTION OF THE DRAWINGS

FIG. 1 is a partial cut-away, perspective view of an MRI system and medical device according to the present invention.

FIG. 2 is a top, perspective view of the medical device shown in FIG. 1.

FIG. 3 is an enlarged, perspective view of an exemplary tracking device used in the medical device shown in FIG. 2.

FIG. 4 is an enlarged, perspective view of an exemplary RF tracking coil and tube used in the tracking device shown in FIG. 3.

FIG. 5 is a graphic representation of the basic pulse sequence diagram used to acquire location data for a MR tracking device.

FIG. 6 is a graphic representation of the pulse sequence diagram in accordance with the present invention.

FIG. 7 is a flow chart of an exemplary algorithm used to practice the system and method of tracking a medical device using MR according to the present invention.

### DETAILED DESCRIPTION OF THE INVENTION

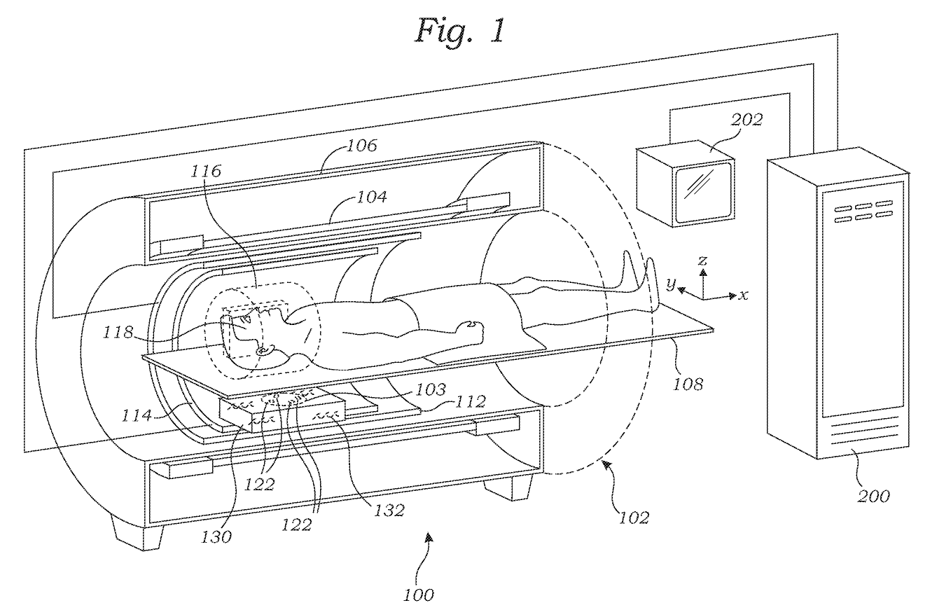

As shown in FIG. 1 the MRI system 100 according to the present invention comprises an MRI machine 102, and a medical device 103 disposed within the bore of the MRI machine 102. The components and operation of the MRI machine are known in the art so only some basic components helpful in the understanding of the system 100 and its operation will be described herein.

The MRI machine 102 typically comprises a cylindrical electromagnet 104 which generates a static magnetic field within the bore 105 of the electromagnet 104. The electromagnet 104 generates a substantially homogeneous magnetic field within the imaging region 116 inside the magnet bore 105. The electromagnet 104 may be housed in a magnet housing 106. A support table 108 is disposed within the bore 105 of the magnet, upon which a patient 110 is placed. The patient is located with the volume of interest 118 within the patient 110 placed within the imaging region 116 of the MRI machine 102.

A set of cylindrical magnetic field gradient coils 112 are also provided within the bore of the magnet 104. The gradient coils 112 also surround the patient 1 10. The gradient coils 112 generate magnetic field gradients of predetermined magnitudes and at predetermined times, and in three mutually orthogonal directions within the magnet bore 105. An RF transmitter coil 114 surrounds a region of interest within the bore 105 which defines the imaging region 116. The RF transmitter coil 114 emits RF energy in the form of a magnetic field into the imaging region 116, including into the volume of interest 118 within the patient 110.

The RF transmitter coil 114 can also receive the MR response signal emitted by the spins which are resonating as a result of the RF pulse generated by the RF transmitter coil 114. The MR response signal that is received by the RF coil 114 is amplified, conditioned and digitized into data using an image processing system 200, as is known by those of ordinary skill in the art. The image processing system 200 further processes the digitized data using known computational methods, including fast Fourier transform (FFT), into an array of image data. The image data is then displayed on a monitor 202, such as a computer CRT, LCD display or other suitable display.

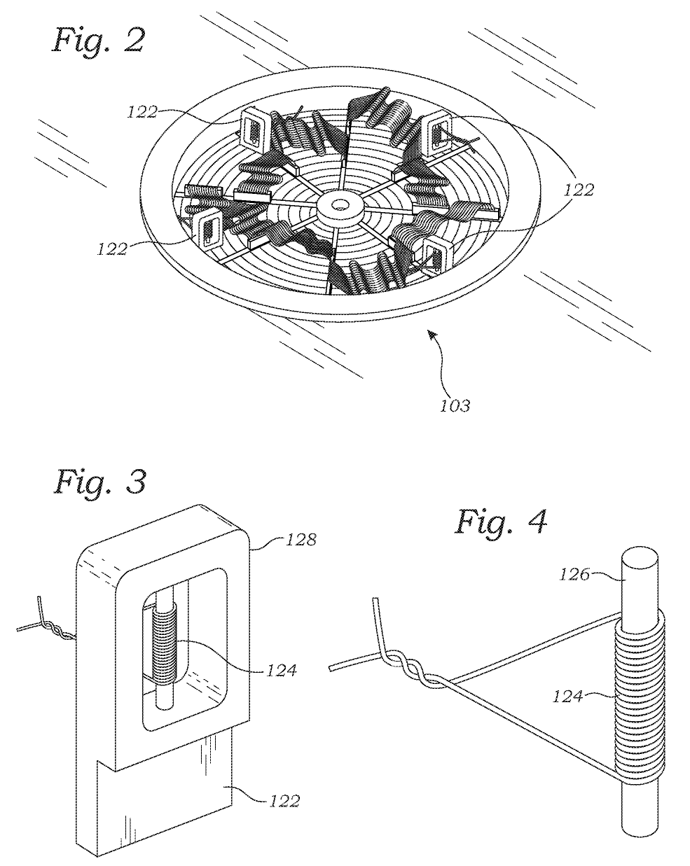

The medical device 103 is placed also within the imaging region 116 of the MRI machine 102. In the example shown in FIG. 1, the medical device 103 is an ultrasonic ablation instrument used for ablating tissue such as fibroids, cancerous or non-cancerous tissue, breaking up occlusion within vessels, or performing other treatment of tissues on or within the patient 110. Referring to FIG. 2, the medical device 103 has one or more tracking devices 122, in this case four tracking devices 122 are utilized. The tracking devices 122 are located at known positions on the medical device 103.

It should be understood that the medical device 103 can be any type of medical instrument including without limitation, a needle, catheter, guidewire, radiation transmitter, endoscope, laparoscope, or other instrument. In addition, the medical device 103 can be configured for placement external of the body of the patient 110, or for insertion into the patient, such as with a catheter. In the case of medical device intended for insertion into the patient 110, the tracking device(s) 122 can be located on the medical device such that the tracking device(s) 122 is inserted into the body of the patient 110, such as at the tip of a catheter or needle.

As shown in more detail in FIGS. 3 and 4, the tracking devices 122 comprise an RF tracking coil 124 wound around a tube 126. The RF coil 124 is formed of a conductive wire such as copper. The tube 126 may be formed of any suitable material, including glass, plastic polymers, etc. The tube 126 may be filled with oil, water or any other MR sensitive matter. The tube 126 and RF coil 124 are attached to a tracking device housing 128, formed of plastic or other suitable electrically non-conductive material.

In order to improve the transmission of the ultrasonic energy from the medical device 103, the medical device may be placed in a transducer housing 130 which is filled with an ultrasound transmission medium 132 such as de-gassed water, gel or other suitable medium.

The RF tracking coils 124 are electrically connected to the image processing system 200. The MR response signal received by the tracking devices 122 are amplified, conditioned and digitized into data using the image processing system 200. The image processing system 200 which processes the signal similar to the processing of the signal received by the RF coil 114, as described above. Alternatively, the RF tracking coils may be electrically connected to a second image processing system (not shown) which is separate from the image processing system 200. The second image processing system processes the MR response signal received by tracking devices 122 similarly to the image processing system 200. The second image processing system may transmits the resulting tracking data to the first image processing system, where it can be superimposed onto the display 202 with the image from the MRI system (i.e. produced from the signal received by the RF transmitting coil 114), or it can be displayed on a separate display from the display 202.

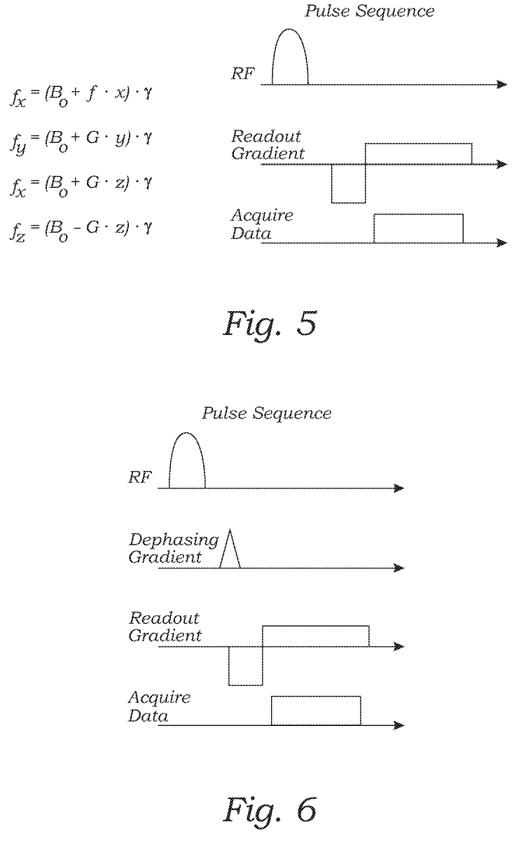

The position and orientation of the medical device 103 within the imaging region 116 relative to the patient volume of interest may be determined using the MRI machine 102 to image the volume of interest, and the tracking devices 122 to determine the location and orientation of the medical device 103. In operation, the MRI machine 102 activates the gradient coils 112, the RF transmitting coil 114 and the RF tracking coils using the pulse sequence diagram (PSD) as shown in FIG. 6. The presented pulse sequence diagram shown in FIG. 6. is conceptually only. The sequence should be repeated at least three times, each in a different readout gradient direction i.e. X, Y and Z to determine the tracking device projection on each axis. For each readout gradient direction the dephasing gradient direction selected to be perpendicular to the readout gradient direction and preferably along the longest axis of the MR sensitive material causing the interference. The PSD of FIG. 6 can be compared to the typical PSD which is shown in FIG. 5. In the modified PSD of FIG. 6, a dephasing gradient is applied perpendicular to readout gradient before the MR response signal is received by the RF tracking coils. The dephasing gradient is preferably applied along the longest axis of the material causing an interfering signal. In the present example, the volume of water, or other medium 132 surrounding the medical device 103, is the major source of interference. Accordingly, the longest axis of the material which may cause interference is the long axis of the volume of medium 132. The dephasing gradient strongly attenuates the interfering signal produced by the medium and any other source of interfering signal emanating from outside the close vicinity of the tracking devices 122. As explained above, the effect on the primary MR response signal produced by the material in the close vicinity of each RF tracking coil 124 is extremely small as compared to the attenuation effect on the interfering signal received by each RF tracking coil 124. In other words, the signal-to-noise ratio (SNR) of the primary MR response signal received by the RF tracking coils 124 is substantially increased by using the modified PSD.

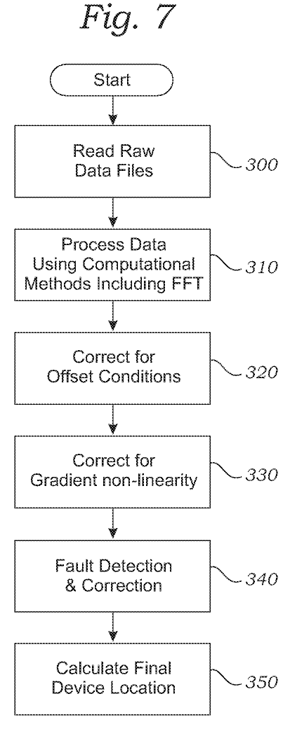

The RF response signal received by each of the RF tracking coils 124 is then processed by the image processing system 200 using computational techniques generally known in the art. For example, referring to FIG. 7, a flow chart of an exemplary algorithm according to the present invention is shown. First, at step 300, the raw data files are read and arranged for each tracking device 122. The raw data comprises the conditioned and digitized data from the MR response signal received by each RF tracking coil 124. The raw data is processed using computational methods including FFT, to calculate the location of each tracking device 122 in the MR coordinates. The location information is then corrected to account for static magnetic field offset conditions (Bo), table position and for gradient non-linearity at steps 320 and 330, respectively. At step 340, the algorithm detects faults and errors in the individual and in the set of tracking devices' 122 location. The error detection is based on two different methods, SNR of each tracking device 122 and the distances between the devices. If the SNR of an individual tracking device 122 is found to be below a preset value the specific tracking device data is ignored and if the distance of a tracking device to other devices deviates from the known location of the tracking devices 122 the tracking device data is ignored also. At step 350, the final location of the medical device is calculated taking into account only the data from valid tracking devices. If not enough tracking devices' data are valid to determine the medical device location a new tracking scan is performed. This result is used to determine the new location of the medical device 103. The image on the display 202 is then updated based on the new tracking device location data. This process is repeated so long as tracking of the medical device 103 is needed.

While embodiments of the present invention have been shown and described, various modifications may be made without departing from the scope of the present invention. The invention, therefore, should not be limited, except to the following claims, and their equivalents.

1. A system for tracking the location of a medical device within an MRI machine, comprising:
an MRI machine;
a medical device for treating or diagnosing a patient;
a tracking device attached to said medical device, said tracking device adapted to receive an MR response signal, having a readout gradient, generated by material in a close vicinity of said tracking device when said material is excited by said MR machine;
wherein said MRI machine is adapted to apply a dephasing gradient substantially perpendicular to said readout gradient before said MR response signal is received by the tracking device.

2. The system of claim 1, wherein said MRI machine comprises a magnetic gradient generator for generating a magnetic field of varying amplitude in a selected number of dimensions, and said magnetic gradient generator applies said dephasing gradient.

3. The system of claim 1, wherein said tracking device comprises at least one RF tracking coil.

4. The system of claim 1, wherein said tracking device comprises three or more RF coils.

5. The system of claim 1, further comprising an image processing system operably coupled to at least one of said MRI machine and said tracking device.

6. The system of claim 5 wherein said image processing system is operably coupled to a display, and said image processing system is adapted to display an image of a region of interest of the patient and superimpose an illustration of the medical device representing the location of said medical device onto said image of a region of interest of the patient.

8. The system of claim 1, wherein interfering material outside the close vicinity of said tracking device produces an interfering MR response signal which is attenuated by said dephasing gradient, said interfering material having a largest dimension substantially along the axis of the dephasing gradient.

9. A system for tracking the location of a medical device within an MRI machine, comprising:
an MRI machine comprising;
a magnet for applying a substantially uniform magnetic field throughout an imaging region within said MRI machine;
a magnetic gradient generator for generating a magnetic field of varying amplitude in a selected number of dimensions within said imaging region;
an RF transmitter for transmitting RF energy of a selected pulse sequence within said imaging region;
an RF receiver for receiving a magnetic resonance response signal emitted from resonating nuclei;
an image processing system operably coupled to said RF receiver, said image processing system adapted to process said magnetic resonance response signal into spatial location an image data; and
a display operably coupled to said image processing system for displaying the image represented by said image data;
a medical device for treating or diagnosing a patient;
a tracking device attached to said medical device, said tracking device adapted to receive an MR response signal, having a readout gradient, generated by material in a close vicinity of said tracking device which is excited by said MR machine, said tracking device operably coupled to said image processing system, said image processing system adapted to process said; and
wherein said MRI machine is adapted to apply a dephasing gradient substantially perpendicular to said readout gradient before said RF response signal is received by the tracking device.

10. A method of tracking the location of a medical device within an imaging region of an MRI machine, comprising the following steps:
a) providing a medical device within the imaging region of the MRI machine, said medical device having a tracking device attached thereto, said tracking device adapted to receive an MR response signal generated by material in a close vicinity of said tracking device when said material is excited by said MRI machine;
b) transmitting an RF pulse into said imaging region and applying a magnetic field gradient to the imaging region such that material in the close vicinity of said tracking device emits an MR response signal;
c) applying a dephasing gradient substantially perpendicular to said readout gradient;
d) receiving said MR response signal with said tracking device; and
e) determining the location of said tracking device using said received MR response signal.

11. The method of claim 10 wherein said step of determining the location of said tracking device comprises conditioning the received MR response signal, digitizing the signal into data, and processing the digitized data computational methods including FFT into image data representing the location of said tracking device.

12. The method of claim 10 wherein said tracking device comprises at least one RF tracking coil.

13. The method of claim 10 wherein said tracking device comprises more than one RF tracking coil.

14. The method of claim 11 wherein said step of processing said digitized data further comprises correcting for offset conditions and correcting for gradient non-linearity.

15. The method of claim 10 further comprising:
acquiring a patient image of a region of interest of a patient within the imaging region and displaying the patient image on a display; and
indicating the location of said medical device on the patient image on said display.

16. The method of claim 14 further comprising:
f) acquiring a patient image of a region of interest of a patient within the imaging region and displaying the patient image on a display;
g) indicating the location of said medical device on the patient image on said display;
h) repeating steps a)-e) to determine an updated location of said medical device indicating an updated location of said medical device on said display.

17. The method of claim 10 wherein interfering material outside the close vicinity of said tracking device produces an interfering RF response signal which is attenuated by said dephasing gradient, and said dephasing gradient is applied substantially along the axis most closely corresponding to the large axis of said interfering material.

18. The method of claim 13 further comprising the steps of:
measuring the amplitude of the MR response signal received by each said RF coil; and
if the amplitude of the MR response signal received by any RF coil is below a threshold value, ignoring the data from such RF coil in said step of determining the location of said tracking device.

19. The method of claim 18 further comprising the steps of:
determining the detected location of each RF coil using said MR response signal;
comparing the distance between the detected location of each RF coil and at least one of the other RF coils to a known predetermined distance between such RF coils;
if the distance between the detected location of an RF coil and one of the other RF coils deviates from said know predetermined distance between such RF coils, ignoring the data from such RF coils in said step of determining the location of said tracking device.

20. The method of claim 19 further comprising the step of:
if not enough RF coils yield valid data to determine tracking device location, performing a new tracking scan.
